(19) United States
(12) Patent Application Publication
(10) Pub. No.: US 2012/0065151 A1
(43) Pub. Date: Mar. 15, 2012

(54) Substances Mixture, Use and Infusion Solution

(76) Inventor: Marianne Broendlund, Harlev (DK)

(21) Appl. No.: 13/230,651

(22) Filed: Sep. 12, 2011

(30) Foreign Application Priority Data
Sep. 13, 2010 (DE) 10 2010 045 185.1

(51) Int. Cl. A61K 31/7004; A61P 35/02; A61P 3/10; A61P 35/04; A61P 9/00; A61P 3/06; A61P 19/08; A61P 29/00; A61P 35/00; A61P 37/06

(52) U.S. Cl. 514/23

(57) ABSTRACT

The present disclosure relates to a substance mixture, wherein the substance mixture contains ribose, alanine, nicotinic acid and ascorbic acid.

### CROSS REFERENCE TO RELATED APPLICATION

This application claims priority to German Patent Application No. 10 2010 045 185.1, entitled “Substances Mixture, Use and Infusion Solution,” filed Sep. 13, 2010, which is hereby incorporated by reference in its entirety for all purposes.

### TECHNICAL FIELD

The present disclosure relates to a composition, to the use of a composition for manufacturing a medicine or a food supplement and to an infusion solution.

### BACKGROUND AND SUMMARY

It is the goal of the present disclosure to provide a composition, for example as a medicament or a food supplement, the composition comprising natural ingredients which can be used in the treatment of a plurality of diseases. This is achieved by a composition wherein the composition contains a combination mixture including ribose, alanine, nicotinic acid and ascorbic acid. This may also be achieved by a solution wherein the solution contains a liquid composition in a twenty-five-fold or fifty-fold dilution in an aqueous solution of water or isotonic saline. The solution may be used as a drinking solution or an infusion solution. This is also achieved by a use of a composition to assist in the treatment of one or more diseases.

Accordingly, the present disclosure relates to a composition containing a combination mixture of ribose, alanine, nicotinic acid and ascorbic acid. The combination mixture in accordance with the present disclosure serves the provision of a medicament and/or of a drug or food supplement which preferably comprises only natural ingredients.

In an embodiment, the ribose used is D-ribose. This represents an important module in the human body, for example in the RNA backbone and as a component of adenosine, and thus of ATP, ADP, AMP or cAMP.

In an embodiment, the alanine used is racemic (DL)-α-alanine. Alanine can serve as a constituent of solutions, such as infusion solutions, for parenteral nutrition.

In an embodiment, the ascorbic acid used is L-ascorbic acid which is also known under the common name of vitamin C and which strengthens the body's defenses. In one example, the medicament may include each of ribose as D-ribose, alanine as DL-α alanine, and ascorbic acid as L-ascorbic acid. However, in another example, only ribose may be included as D-ribose (and alanine may not be included as DL-α alanine, and ascorbic acid may not be included as L-ascorbic acid). In another example, only alanine may be included as DL-α alanine (and ribose may not be included as D-ribose and ascorbic acid may not be included as L-ascorbic acid). In still another example, only ascorbic acid may be included as L-ascorbic acid (and alanine may not be included as DL-α alanine and ribose may not be included as D-ribose). Still other combinations may be possible. For example, the composition may include D-ribose, and DL-α alanine, but not L-ascorbic acid. Alternatively, the composition may include D-ribose, L-ascorbic acid but not DL-α alanine. Further still, the composition may include DL-α alanine and L-ascorbic acid, but not D-ribose.

In an embodiment, the composition in accordance with the present disclosure additionally contains thiamine. Thiamine is also known under the common name of vitamin B1 and strengthens the patient's defenses and nervous system.

Nicotinic acid is also known under the common name of vitamin B5 and plays an important role in the metabolism.

A composition in accordance with the present disclosure therefore represents a new combination of active ingredients in a composition which strengthens different bodily functions and can successfully serve the treatment and the assisting in treatment of a plurality of diseases when employed in a suitable administration and dosage.

In an embodiment, the composition in accordance with the present disclosure is a solid composition which contains the components as solids. Ribose, alanine, nicotinic acid and ascorbic acid as well as the optionally present thiamine are typically present as a solid white powder at room temperature.

In an embodiment, the solid composition in accordance with the present disclosure is a powdery mixture of the powdery constituents ribose, alanine, nicotinic acid and ascorbic acid as well as optionally thiamine. In an embodiment, the solid composition in accordance with the present disclosure substantially or only comprises these constituents.

In an embodiment, the solid composition in accordance with the present disclosure is provided in a specific dosage in separate containers. Suitable containers include, for example, capsules, paper bags, paper sachets, plastic shells or plastic tubes. Since the solid composition in accordance with the present disclosure is light-sensitive, it is moreover recommended that the container protects the composition from light incident from the outside. Particularly suitable containers therefore also include those made of tinted glass.

In an embodiment, a container contains exactly one dosage unit which contains between about 40 g and about 250 g ribose (e.g., plus or minus 10% of the indicated weight range), between about 5 g and about 50 g alanine (e.g., plus or minus 10% of the indicated weight range), between about 3 g and about 30 g nicotinic acid (e.g., plus or minus 10% of the indicated weight range) and between about 5 g and about 75 g ascorbic acid (e.g., plus or minus 10% of the indicated weight range). This represents a suitable dosage with respect to the substance quantities and in particular to the substance quantity ratios in a further use as or for the manufacture of a medicine, a food supplement, and/or a solution for example by dissolving and optionally diluting.

In one example, the quantity of ribose within a dosage unit is between about 60 g and about 200 g, the quantity of alanine within a dosage unit is between about 10 g and about 40 g, the quantity of nicotinic acid within a dosage unit is between about 5 g and about 25 g, and the quantity of ascorbic acid within a dosage unit is between about 10 g and about 50 g.

If the solid composition in accordance with the present disclosure should additionally contain thiamine, a dosage unit in an embodiment can contain between about 2 g and about 8 g, for example, between about 4 g and about 6 g thiamine.

In an embodiment, a dosage unit of a composition in accordance with the present disclosure contains between about 65 g and about 80 g ribose, for example between about 70 g and about 74 g ribose, between about 15 g and about 25 g alanine, for example between about 17 g and about 21 g alanine, between about 10 g and about 15 g nicotinic acid, for example between about 11 g and about 13 g nicotinic acid, as well as between about 30 g and about 40 g ascorbic acid, for example between about 34 g and about 38 g ascorbic acid. Such a composition is in particular suitable for providing a medicine and/or a food supplement for the treatment or for the assisting in treatment of different types of cancer, of lymphogenic leukemia, of lupus erythematodes or of diabetes mellitus. This solid composition in accordance with the present disclosure will be called solid composition A in the further text.

In another embodiment, a dosage unit of a composition in accordance with the present disclosure contains between about 55 g and about 65 g ribose, for example between about 58 g and about 62 g ribose, between about 5 g and about 15 g alanine, for example between about 8 g and about 12 g alanine, between about 12 g and about 18 g nicotinic acid, for example between about 13 g and about 17 g nicotinic acid, as well as between about 5 g and about 15 g ascorbic acid, for example between about 8 g and about 12 g ascorbic acid. Such a composition is in particular suitable for providing a medicine and/or a food supplement for the treatment or the assisting in treatment of coronary heart disease, coronary insufficiencies and lipid metabolic disorders. This solid composition in accordance with the present disclosure will be called solid composition B in the further text.

In yet another embodiment, a dosage unit of a composition in accordance with the present disclosure contains between about 55 g and about 65 g, or between about 58 g and about 62 g ribose, between about 15 g and about 25 g, or between about 17 g and about 21 g alanine, between about 8 g and about 15 g, or between about 9 g and about 13 g nicotinic acid, between about 40 g and about 55 g, or between about 46 g and about 50 g ascorbic acid and between about 3 g and about 7 g, or between about 4 g and about 6 g thiamine. Such a composition is in particular suitable for providing a medicine and/or a food supplement for the treatment or the assisting treatment of degenerative bone diseases, rheumatoid arthritis as well as of bone metastases of tumors. This solid composition in accordance with the present disclosure will be called solid composition C in the further text.

In an embodiment, the composition in accordance with the present disclosure is in a liquid form which contains the constituents in aqueous solution. Ribose, alanine, nicotinic acid and ascorbic acid as well as the optionally present thiamine are easily soluble in water whereby they can be dissolved without problem in pure water or in aqueous solutions at room temperature.

Since the liquid composition in accordance with the present disclosure is light-sensitive, in one embodiment, the components may be protected from light incident from the outside. Particularly suitable containers, for example, include ampoules made from tinted glass.

In an embodiment, a liquid composition in accordance with the present disclosure contains the constituents in a solution of a physiological saline solution (also isotonic saline solution or 0.9% saline solution). The use of other isotonic solutions is also conceivable. The liquid composition may be provided as a solution, such as a drinking solution or an infusion solution.

Provision can be made in an embodiment that a liquid composition in accordance with the present disclosure substantially or exclusively comprises the constituents ribose, alanine, nicotinic acid, ascorbic acid, optionally thiamine, and water or physiological saline solution.

In one embodiment, a liquid composition in accordance with the present disclosure contains between 40 g/l and 250 g/l ribose, between about 5 g/l and about 50 g/l alanine, between about 3 g/l and about 30 g/l nicotinic acid and between about 5 g/l and about 75 g/l ascorbic acid. This represents a suitable dosage with respect to the concentrations and in particular with respect to the concentration ratios on a use as a medicine and/or as a food supplement or on a further use for manufacturing a medicine and/or a food supplement by dilution, for example.

In one example, the concentration range for ribose is between about 60 g/l and about 200 g/l. The concentration range for alanine is between about 10 g/l and about 40 g/l, the concentration range for nicotinic acid is between about 5 g/l and about 25 g/l, and the concentration range for ascorbic acid is between about 10 g/l and about 50 g/l.

If the liquid composition in accordance with the present disclosure should additionally contain thiamine, its concentration in an embodiment amounts to between about 2 g/l and about 8 g/l, for example, between about 4 g/l and about 6 g/l.

In an embodiment, a liquid composition in accordance with the present disclosure contains between about 65 g/l and about 80 g/l ribose, for example between about 70 g/l and about 74 g/l ribose, between about 15 g/l and about 25 g/l alanine, for example between about 17 g/l and about 21 g/l alanine, between about 10 g/l and about 15 g/l nicotinic acid, for example between about 11 g/l and about 13 g/l nicotinic acid as well as between about 30 g/l and about 40 g/l ascorbic acid, for example between about 34 g/l and about 38 g/l ascorbic acid. Such a composition is in particular suitable for treating, providing a medicine and/or a food supplement, in particular a solution, such as an infusion solution, for the treatment or for the assisting in treatment of different types of cancer, of lymphogenic leukemia, of lupus erythematodes or of diabetes mellitus. This liquid composition in accordance with the present disclosure will be called liquid composition A in the further text.

In an embodiment, a liquid composition in accordance with the present disclosure contains between about 55 g/l and about 65 g/l ribose, for example between about 58 g/l and about 62 g/l ribose, between about 5 g/l and about 15 g/l alanine, for example between about 8 g/l and about 12 g/l alanine, between about 12 g/l and about 18 g/l nicotinic acid, for example between about 13 g/l and about 17 g/l nicotinic acid, as well as between about 5 g/l and about 15 g/l ascorbic acid, for example between about 8 g/l and about 12 g/l ascorbic acid. Such a composition is in particular suitable for treating, providing a medicine and/or a food supplement, for example as an infusion solution, for the treatment or the assisting in treatment of coronary heart disease, coronary insufficiencies and lipid metabolic disorders. This liquid composition in accordance with the present disclosure will be called liquid composition B in the further text.

In an embodiment, a liquid composition in accordance with the present disclosure contains between about 55 g/l and about 65 g/l ribose, for example between about 58 g/l and about 62 g/l ribose, between about 15 g/l and about 25 g/l alanine, for example between about 17 g/l and about 21 g/l alanine, between about 8 g/l and about 15 g/l nicotinic acid, for example between about 9 g/l and about 13 g/l nicotinic acid, between about 40 g/l and about 55 g/l ascorbic acid, for example between about 46 g/l and about 50 g/l ascorbic acid and between about 3 g/l and about 7 g/l thiamine, preferably between about 4 g/l and about 6 g/l thiamine. Such a composition is in particular suitable for treating, providing a medicine and/or a food supplement, for example, as an infusion solution, for the treatment or the assisting in treatment of degenerative bone diseases, rheumatoid arthritis as well as of bone metastases of tumors. This liquid composition in accordance with the present disclosure will be called liquid composition C in the further text.

In an embodiment, the liquid composition in accordance with the present disclosure is a concentrate. This can be further diluted, for example with water or physiological saline solution, to obtain a medicine, solution, and/or food supplement ready for administration. The above-named mixture ratios in the concentrate are already selected in this respect so that the desired substance quantity ratios are achieved by a simple dilution.

In another embodiment, the liquid composition in accordance with the present disclosure is a medicine, solution, and/or a food supplement ready for administration, the food supplement used for general nutrition or health related purposes. This can be administered intravenously, for example, in a suitable dosage to treat a patient. Suitable dosages comprise between about 5 ml and about 15 ml, preferably between about 8 ml and about 12 ml. The intravenous administration of exactly 10 ml of the liquid composition in accordance with the present disclosure is particularly preferred.

In addition to the administration of the liquid composition in accordance with the present disclosure, the preceding or subsequent, preferably intravenous, administration of a homeopathic active ingredient can establish or amplify the effect and the healing power of a composition in accordance with the present disclosure.

Preferred homeopathic active ingredients for this application include active ingredients selected from the group Magnesium phosphoricum, Natrium pyruvicum, Natrium diethyloxalaceticum, Acidum citricum, Acidum cis-aconiticum, Barium oxalsuccinicum, Acidum a-ketoglutaricum, Acidum succinicum, Acidum fumaricum, Acidum DL-malicum, Strychnos nux-vomica, Ubidecarenonum, Natrium pyruvicum, Strychnos ignatii, Hepar suis, Silybum marianum, Ren suis, Calendula officinalis, Atropa bella-donna, Acontium napellus, Bellis perennis, Hypericum perforatum, Echinacea, Echinacea purpurea, Symphytum officinale, Matricaria recutita, Achillea millefolium, Mercurius solubilis Hahnemanni, Hepar sulfuris, Hamamelis virginiana, Arnica montana and Zincum valerianicum.

The present disclosure further relates to a method for providing a liquid composition in accordance with the present disclosure, wherein a solid medicament in accordance with the present disclosure is dissolved in water, in a physiological saline solution or in another physiologically compatible aqueous solution.

In an embodiment, this solution procedure takes place at room temperature (approx. 21° C.) while carefully stirring and under sterile conditions.

In an embodiment, a dosage unit of a solid composition in accordance with the present disclosure is dissolved in between 500 ml and 1500 ml, or between 800 ml and 1200 ml, or exactly 1000 ml water, physiological saline solutions or another physiologically compatible aqueous solution.

In an embodiment, one or several homeopathic active ingredients are added to the solution before, during and/or after the solution procedure.

The present disclosure furthermore relates to a solution which contains the ingredients of a liquid composition in accordance with the present disclosure in a twenty-fold to sixty-fold dilution. The solution may be a drinking solution or an infusion solution. A solution in accordance with the present disclosure preferably contains these ingredients in twenty-five fold or fifty-fold dilution. A physiological saline solution (also isotonic saline solution or 0.9% saline solution) preferably serves as the basis for the solution. The use of another infusion solution basis is also conceivable. The infusion solutions are suitable for parenteral administration to a patient.

Depending on the desired treatment intensity and on the condition of the patient, a treating physician can adapt the degree of dilution of the solution within the limits of the present disclosure.

In an embodiment, a solution in accordance with the present disclosure which contains the ingredients of a liquid composition in accordance with the present disclosure in twenty-five-fold dilution contains between about 1.5 g/l and about 10 g/l ribose, between about 0.2 g/l and about 2 g/l alanine, between about 0.10 g/l and about 1 g/l nicotinic acid, and between about 0.2 g/l and about 3 g/l ascorbic acid and a solutionin accordance with the present disclosure which contains the ingredients of a liquid composition in accordance with the present disclosure in fifty-fold dilution contains between about 0.7 g/l and about 5 g/l ribose, between about 0.1 g/l and about 1 g/l alanine, between about 0.05 g/l and about 0.5 g/l nicotinic acid, and between about 0.1 g/l and about 1.5 g/l ascorbic acid. This represents a suitable concentration ratio for a treatment.

For example, a concentration range for ribose at twenty-five-fold dilution is between about 2.5 g/l and about 8 g/l and at fifty-fold dilution between about 1.2 g/l and about 4 g/l. An example concentration range for alanine at twenty-five-fold dilution is between about 0.5 g/l and about 1.5 g/l and at fifty-fold dilution between about 0.2 g/l and about 0.8 g/l. An example concentration range for nicotinic acid at twenty-five-fold dilution is between about 0.2 g/l and about 1 g/l and at fifty-fold dilution between about 0.1 g/l and about 0.5 g/l. An example concentration range for ascorbic acid at twenty-five-fold dilution is between about 0.4 g/l and about 2 g/l and at fifty-fold dilution between about 0.2 g/l and about 1 g/l.

If the solution in accordance with the present disclosure should additionally contain thiamine, its concentration in an embodiment at twenty-five-fold dilution amounts to between about 0.1 g/l and about 0.3 g/l, for example between about 0.15 g/l and about 0.25 g/l, and at fifty-fold dilution between about 0.05 g/l and about 0.2 g/l.

In an embodiment, a solution in accordance with the present disclosure contains at twenty-five fold dilution between about 2.6 g/l and about 3.2 g/l, for example between about 2.8 g/l and about 3 g/l ribose, between about 0.6 g/l and about 1 g/l, for example between about 0.7 g/l and about 0.9 g/l alanine, between about 0.4 g/l and about 0.6 g/l, for example about 0.5 g/l nicotinic acid, as well as between about 1.2 g/l and about 1.6 g/l, for example between about 1.3 g/l and about 1.5 g/l ascorbic acid, and at fifty-fold dilution between about 1.3 g/l and about 1.6 g/l, for example between about 1.4 g/l and about 1.5 g/l ribose, between about 0.3 g/l and about 0.5 g/l alanine, between about 0.2 g/l and about 0.3 g/l nicotinic acid, and between about 0.6 g/l and about 0.8 g/l ascorbic acid. Such a solution is in particular suitable for the treatment or the assisting in treatment of different types of cancer, of lymphogenic leukemia, of lupus erythematodes, or diabetes mellitus. The solution in accordance with the present disclosure will be called infusion A in the further text.

In an embodiment, a solutionin accordance with the present disclosure contains at twenty-five fold dilution between about 2.2 g/l and about 2.6 g/l, for example between about 2.3 g/l and about 2.5 g/l ribose, between about 0.2 g/l and about 0.6 g/l, for example between about 0.3 g/l and about 0.5 g/l alanine, between about 0.5 g/l and about 0.7 g/l, for example about 0.6 g/l nicotinic acid, as well as between about 0.2 g/l and about 0.6 g/l, for example between about 0.3 g/l and about 0.5 g/l ascorbic acid, and at 50-fold dilution between about 1.1 g/l and about 1.3 g/l ribose, between about 0.1 g/l and about 0.3 g/l alanine, between about 0.2 g/l and about 0.4 g/l nicotinic acid, as well as between about 0.1 g/l and about 0.3 g/l ascorbic acid. Such a solution is in particular suitable for the treatment or assisting in treatment of coronary heart diseases, coronary insufficiencies and lipid metabolic disorders. The solution in accordance with the present disclosure will be called infusion B in the further text.

In an embodiment, a solution in accordance with the present disclosure contains at twenty-five fold dilution between about 2.2 g/l and about 2.6 g/l, for example between about 2.3 g/l and about 2.5 g/l ribose, between about 0.6 g/l and about 1 g/l, for example between about 0.7 g/l and about 0.9 g/l alanine, between about 0.3 g/l and about 0.6 g/l, for example between about 0.4 g/l and about 0.5 g/l nicotinic acid, between about 1.6 g/l and about 2.2 g/l, for example between about 1.8 g/l and about 2 g/l ascorbic acid, as well as between about 0.1 g/l and about 0.3 g/l, preferably about 0.2 g/l thiamine, and at fifty-fold dilution between about 1.1 g/l and about 1.3 g/l ribose, between about 0.3 g/l and about 0.5 g/l alanine, between about 0.1 g/l and about 0.3 g/l nicotinic acid, between about 0.8 g/l and about 1.1 g/l, for example between about 0.9 g/l and about 1 g/l, ascorbic acid, as well as between about 0.05 g/l and about 0.15 g/l thiamine. Such a solutionis in particular suitable for the treatment or the assisting in treatment of degenerative bone diseases, rheumatoid arthritis as well as of bone metastases of tumors. This solution in accordance with the present disclosure will be called infusion C in the further text.

In an embodiment, a solution in accordance with the present disclosure contains at least one homeopathic active ingredient. Example homeopathic active ingredients in this respect include active ingredients selected from the group Magnesium phosphoricum, Natrium pyruvicum, Natrium diethyloxalaceticum, Acidum citricum, Acidum cis-aconiticum, Barium oxalsuccinicum, Acidum a-ketoglutaricum, Acidum succinicum, Acidum fumaricum, Acidum DL-malicum, Strychnos nux-vomica, Ubidecarenonum, Natrium pyruvicum, Strychnos ignatii, Hepar suis, Silybum marianum, Ren suis, Calendula officinalis, Atropa bella-donna, Acontium napellus, Bellis perennis, Hypericum perforatum, Echinacea, Echinacea purpurea, Symphytum officinale, Matricaria recutita, Achillea millefolium, Mercurius solubilis Hahnemanni, Hepar sulfuris, Hamamelis virginiana, Arnica montana and Zincum valerianicum.

This addition of the homeopathic active ingredients can establish or amplify the effect and the healing power of a solution in accordance with the present disclosure.

The present disclosure furthermore relates to a method for providing a solution in accordance with the present disclosure by dilution of a liquid composition in accordance with the present disclosure in a base solution such as preferably a physiological saline solution.

In an embodiment, about 10 ml (between about 8 ml and about 12 ml) of a liquid composition in accordance with the present disclosure is diluted in either about 250 ml or about 500 ml of a solution basis. A physiological saline solution (also isotonic saline solution or 0.9% saline solution) preferably serves as the basis for the solution. The use of another infusion solution basis is also conceivable.

In an embodiment, one or several homeopathic active ingredients are added to the solution before and/or during and/or after the solution procedure.

The present disclosure further relates to the use of a composition in accordance with the present disclosure for manufacturing a medicine and/or a food supplement, for example a solution, such as an infusion solution or drinking solution in accordance with the present disclosure.

Such a medicine, food supplement or infusion solution, prepared in accordance with the present disclosure can be used in the sole treatment or as a complement to the classical medicine treatment. The administration strengthens the body and the immune system, increases the well-being of the patient and thus promotes healing.

The use of the composition may be, for example, for manufacturing a medicine and/or a food supplement, and/or an infusion solution, for the treatment of, or assisting in the treatment of diseases selected from the group cancer, bone metastases, lymphogenic leukemia, lupus erythematodes, diabetes mellitus, coronary heart diseases, coronary insufficiency, lipid metabolic disorders, degenerative bone diseases and rheumatoid arthritis.

An embodiment relates to the use of a composition A in accordance with the present disclosure for manufacturing an infusion A in accordance with the present disclosure for the treatment or assisting in the treatment of different types of cancer, of lymphogenic leukemia, of lupus erythematodes or diabetes mellitus.

An embodiment relates to the use of a composition B in accordance with the present disclosure for manufacturing an infusion B in accordance with the present disclosure for the treatment or assisting in the treatment of coronary heart diseases, coronary insufficiencies and lipid metabolic disorders.

An embodiment relates to the use of a composition C in accordance with the present disclosure for manufacturing an infusion C in accordance with the present disclosure for the treatment or assisting in the treatment of degenerative bone diseases, rheumatoid arthritis and of bones metastases of tumors.

Further details and advantages of the present disclosure result from the following embodiments.

### Example 1

72 g D-ribose, 19.2 g DL-α-alanine, 12 g nicotinic acid and 36 g L-ascorbic acid are provided in powder form in a packaging unit, for example a coated paper sachet.

They are dissolved in 1 l 0.9% saline solution at 21° C. while stirring carefully under sterile conditions. In this process, a concentrate is created with 72 g/l D-ribose, 19.2 g/L DL-α-alanine, 12 g/l nicotinic acid, 36 g/l L-ascorbinic acid and 9 g/l NaCl.

### Example 2

60 g D-ribose, 10.3 g DL-α-alanine, 15 g nicotinic acid and 10 g L-ascorbic acid are provided in powder form in a packaging unit, for example a coated paper sachet.

They are dissolved, as shown in Example 1, in 1 l isotonic saline solution, with a concentrate being obtained having 60 g/l D-ribose, 10.3 g/l DL-α-alanine, 15 g/l nicotinic acid, 10 g/l L-ascorbic acid and 9 g/l NaCl.

### Example 3

60 g D-ribose, 19.2 g DL-α-alanine, 10.8 g nicotinic acid, 48 g L-ascorbic acid and 4.8 thiamine in powder form are provided in a packaging unit, for example a coated paper sachet.

They are dissolved, as shown in Example 1, in 1 l isotonic saline solution, with a concentrate being obtained having 60 g/l D-ribose, 19.2 g/l DL-α-alanine, 10.8 g/l nicotinic acid, 48 g/l L-ascorbic acid, 4.8 g/l thiamine and 9 g/l NaCl.

### Example 4

A homeopathic active ingredient mixture is produced, with the contents of the following ampoules of the company Heel—Healthcare Designed by Nature being combined in a glass container:

10 ampoules @ 1.1 ml Citric Acid Cycle Heel

10 ampoules @ 1.1 ml Nux vomica-Injeel S

5 ampoules @ 1.1 ml Ubichinon-Injeel forte

5 ampoules @ 1.1 ml Acidum DL-malicum-Injeel,

5 ampoules @ 1.1 ml Natrium pyruvicum-Injeel,

10 ampoules at 1.1 ml Ignatia-Injeel S,

10 ampoules @ 1.1 ml Hepar suis-Injeel,

10 ampoules @ 1.1 ml Carduus marianus-Injeel forte,

10 ampoules @ 1.1 ml Ren suis-Injeel,

5 ampoules @ 5 ml Traumeel S,

5 ampoules @ 5 ml Belladonna-Homaccord, and

10 ampoules @ 1.1 ml Zincum valerianicum-Injeel.

### Example 5

The concentrate of Example 1 is filled in ampoules of tinted glass @ 10 ml in each case. The content of an ampoule is administered to the patient intravenously as a complementary therapy in the treatment of cancer, lymphogenic leukemia, lupus erythematodes and of diabetes mellitus.

### Example 6

In addition to the therapy described in Example 5, 10 ml of the homeopathic active ingredient mixture from Example 4 is subsequently administered intravenously to amplify the healing power of the composition in accordance with the present disclosure of Example 1.

### Example 7

10 ml of the concentrate of Example 1 is added to 250 ml or 500 ml of an infusion solution, preferably 0.9% saline solution. 10 ml of the homeopathic active ingredient mixture of Example 4 is added to the infusion solution.

The created infusion solution is administered to the patient intravenously as a complementary therapy in the treatment of cancer, lymphogenic leukemia, lupus erythematodes and of diabetes mellitus.

### Example 8

The concentrate of Example 2 is filled in ampoules of tinted glass @ 10 ml in each case. The content of an ampoule is administered to the patient intravenously as a complementary therapy in the treatment of coronary heart diseases, coronary insufficiency and of lipid metabolic disorders.

### Example 9

In addition to the therapy described in Example 8, 10 ml of the homeopathic active ingredient mixture from Example 4 is subsequently administered intravenously to amplify the healing power of the composition in accordance with the present disclosure of Example 2.

### Example 10

10 ml of the concentrate of Example 2 is added to 250 ml or 500 ml of an infusion solution, preferably 0.9% saline solution. 10 ml of the homeopathic active ingredient mixture of Example 4 is furthermore added to the infusion solution.

The infusion solution created is administered to the patient intravenously as a complementary therapy in the treatment of coronary heart diseases, coronary insufficiency and of lipid metabolic disorders.

### Example 11

The concentrate of Example 3 is filled in ampoules of tinted glass @ 10 ml in each case. The content of an ampoule is administered to the patient intravenously as a complementary therapy in the treatment of degenerative bone diseases, rheumatoid arthritis and bone metastases.

### Example 12

In addition to the therapy described in Example 11, 10 ml of the homeopathic active ingredient mixture from Example 4 is subsequently administered intravenously to amplify the healing power of the composition in accordance with the present disclosure of Example 3.

### Example 13

10 ml of the concentrate of Example 3 is added to 250 ml or 500 ml of an infusion solution, preferably 0.9% saline solution. 10 ml of the homeopathic active ingredient mixture of Example 4 is furthermore added to the infusion solution.

The infusion solution which is created is administered to the patient intravenously as a complementary therapy in the treatment of degenerative bone diseases, rheumatoid arthritis and bone metastases.

1. A composition, wherein the composition comprises a combination mixture of ribose, alanine, nicotinic acid and ascorbic acid.

2. The composition of claim 1, wherein the ribose is D-ribose; wherein the alanine is DL-α alanine; and wherein the ascorbic acid is L-ascorbic acid.

3. The composition of claim 1, wherein the composition additionally contains thiamine.

4. The composition of claim 1, wherein the composition is in a solid form, and wherein the solid form of the composition contains ribose, alanine, nicotinic acid, ascorbic acid and, optionally thiamine, as solids.

5. The composition of claim 4, wherein the composition is provided in a dosage unit, each dosage unit containing:
between 40 g and 250 g ribose;
between 5 g and 50 g alanine;
between 3 g and 30 g nicotinic acid;
between 5 g and 75 g ascorbic acid; and, optionally,
between 2 g and 8 g thiamine.

6. The composition of claim 3, wherein the composition is in a liquid form, and wherein the liquid form of the composition contains ribose, alanine, nicotinic acid, ascorbic acid and, optionally, thiamine in an aqueous solution.

7. The composition of claim 6, wherein the aqueous solution contains
between 40 g/l and 250 g/l ribose;
between 5 g/l and 50 g/l alanine;
between 3 g/l and 30 g/l nicotinic acid; and
between 5 g/l and 75 g/l ascorbic acid; and, optionally,
between 2 g/l and 8 g/l thiamine.

8. A solution, wherein the solution contains the ingredients of a liquid composition in accordance with claim 7 in a twenty-five-fold or fifty-fold dilution.

9. The solution of claim 8, wherein the solution additionally contains at least one homeopathic active ingredient, and wherein the solution is an infusion solution or a drinking solution.

10. A method, comprising:
administering a composition as a complementary therapy in the treatment of one or more dieases including different types of cancer, lymphogenic leukemia, lupus erythematodes, diabetes mellitus, coronary heart disease, coronary insufficiencies, lipid metabolic disorders, degenerative bone diseases, rheumatoid arthritis, metastases of bone tumors, the composition comprising a combination mixture of ribose, alanine, nicotinic acid, ascorbic acid, and optionally thiamine.

11. The method of claim 10, wherein the composition is a drug or food supplement.

12. The method of claim 10, wherein the composition is in liquid form and is administered as a solution, the solution comprising the liquid composition diluted twenty-five-fold or fifty-fold in water or isotonic saline.

13. The method of claim 10, wherein the complementary therapy includes strengthening of one or more bodily functions.

14. The composition of claim 1, wherein the ribose is D-ribose; wherein the alanine is DL-α alanine; and/or wherein the ascorbic acid is L-ascorbic acid.

15. The composition of claim 5, wherein the composition is provided in a dosage unit, each dosage unit containing:
between 60 g and 200 g ribose;
between 10 g and 40 g alanine;
between 5 g and 25 g nicotinic acid;
between 10 g and 50 g ascorbic acid; and, optionally,
between 4 g and 6 g thiamine.

16. The composition of claim 7, wherein the aqueous solution contains,
between 60 g/l and 200 g/l ribose;
between 10 g/l and 40 g/l alanine;
between 5 g/l and 25 g/l nicotinic acid;
between 10 g/l and 50 g/l ascorbic acid; and, optionally,
between between 4 g/l and 6 g/l thiamine.

17. A method, comprising:
manufacturing a medicine or food supplement, the medicine or food supplement including a composition, the composition comprising a combination mixture of ribose, alanine, nicotinic acid, ascorbic acid, and optionally thiamine.

18. The method of claim 17, wherein composition is a liquid composition, wherein the manufacturing includes providing the medicine or food supplement as a solution containing the liquid composition in a twenty-five-fold or fifty-fold dilution, the liquid composition diluted in water or isotonic saline.

19. The method of claim 18, wherein the liquid composition contains,
between 40 g/l and 250 g/l ribose;
between 5 g/l and 50 g/l alanine;
between 3 g/l and 30 g/l nicotinic acid;
between 5 g/l and 75 g/l ascorbic acid; and, optionally,
between 2 g/l and 8 g/l thiamine.

20. The method of claim 19, wherein the liquid composition further contains at least one homeopathic active ingredient.

21. The composition of claim 1, wherein a relation by weight of ribose, alanine, nicotinic acid and ascorbic acid in the combination mixture is A:B:C:D with,
A between 40 and 250;
B between 5 and 50;
C between 3 and 30; and
D between 5 and 75.

22. The composition of claim 21, wherein A is between 60 and 200, B is between 10 and 40, C is between 5 and 25, and D is between 10 and 50.

23. The composition of claim 22, wherein the combination mixture additionally contains thiamine, and wherein the relation by weight of ribose, alanine, nicotinic acid, ascorbic acid, and thaimine in the combination mixture is A:B:C:D: E with E between 2 and 8.

24. The composition of claim 23, wherein E is between 4 and 6.

25. A solution containing the aqueous solution of claim 7 in a dilution of between 10 and 100.
